SEQUENCE LISTING (13 sequences)

SEQ ID NO: 1 - DNA, 20 bp, Artificial Sequence
  A primer and probe sequence for SARS-CoV-2 RdRp/Helicase
cgcatacagtcttrcaggct

SEQ ID NO: 2 - DNA, 24 bp, Artificial Sequence
  A primer and probe sequence for SARS-CoV-2 RdRp/Helicase
gtgtgatgttgawatgacatggtc

SEQ ID NO: 3 - DNA, 28 bp, Artificial Sequence
  A primer and probe sequence for SARS-CoV-2 RdRp/Helicase
ttaagatgtggtgcttgcatacgtagac

SEQ ID NO: 4 - DNA, 24 bp, Artificial Sequence
  A primer and probe sequence for SARS-CoV-2 RdRp/Helicase
gaccatgtcatwtcaacatcacac

SEQ ID NO: 5 - DNA, 30 bp, Artificial Sequence
  A primer and probe sequence for SARS-CoV-2 Spike (S)
cctactaaattaaatgatctctgctttact

SEQ ID NO: 6 - DNA, 21 bp, Artificial Sequence
  A primer and probe sequence for SARS-CoV-2 Spike (S)
caagctataacgcagcctgta

SEQ ID NO: 7 - DNA, 22 bp, Artificial Sequence
  A primer and probe sequence for SARS-CoV-2 Spike (S)
cgctccagggcaaactggaaag

SEQ ID NO: 8 - DNA, 21 bp, Artificial Sequence
  A primer and probe sequence for SARS-CoV-2 Spike (S)
tacaggctgcgttatagcttg

SEQ ID NO: 9 - DNA, 18 bp, Artificial Sequence
  A primer and probe sequence for SARS-CoV-2 Nucleocapsid (N)
gcgttcttcggaatgtcg

SEQ ID NO: 10 - DNA, 23 bp, Artificial Sequence
  A primer and probe sequence for SARS-CoV-2 Nucleocapsid (N)
ttggatctttgtcatccaatttg

SEQ ID NO: 11 - DNA, 22 bp, Artificial Sequence
  A primer and probe sequence for SARS-CoV-2 Nucleocapsid (N)
aacgtggttgacctacacagst

SEQ ID NO: 12 - DNA, 23 bp, Artificial Sequence
  A primer and probe sequence for SARS-CoV-2 Nucleocapsid (N)
caaattggatgacaaagatccaa

SEQ ID NO: 13 - DNA, 27 bp, Artificial Sequence
  A primer and probe sequence for SARS-CoV-2 RdRp/Helicase
taagatgtggtgcttgcatacgtagac

(19) United States
(12) Patent Application Publication
(10) Pub. No.: US 2023/0349006 A1
(43) Pub. Date: Nov. 2, 2023

(54) COMPOSITIONS AND METHODS FOR DETECTING SARS-CoV-2

(71) Applicant: The University of Hong Kong, Hong Kong (CN)

(72) Inventors: Fuk Woo Jasper Chan, Hong Kong (CN); Kai Wang Kelvin To, Hong Kong (CN); Kwok Yung Yuen, Hong Kong (CN)

(21) Appl. No.: 17/821,411

(22) PCT Filed: Jan. 20, 2021
(86) PCT No.: PCT/CN2021/072838

§ 371 Date: Aug. 22, 2022

Related U.S. Application Data
(60) Provisional application No. 62/980,094, filed on Feb. 21, 2020.

(51) Int. Cl. C12Q 1/70

(52) U.S. Cl. CPC C12Q 1/701; C12Q 2600/112; C12Q 2600/16

(57) ABSTRACT

Provided a nucleic acid probe or primer for the detection of SARS-CoV-2 RdRp/Helicase, Spike (S) or Nucleocapsid (N), and its use in a method of detecting SARS-CoV-2 in a sample. The nucleic acid probe or primer is consisting of a nucleic acid sequence of any of SEQ ID NOs: 1-12.

This international patent application claims the benefit of the filing date of U.S. Provisional Patent Application No. 62/980,094 filed on Feb. 21, 2020, the entire content of which is incorporated by reference for all purpose.

### FIELD OF THE INVENTION

The present invention is generally in the field of detecting SARS-CoV-2.

### BACKGROUND OF THE INVENTION

On 31 Dec. 2019, the World Health Organization was informed of a cluster of cases of pneumonia of unknown etiology in Wuhan, China. Subsequent investigations identified a new coronavirus, now named as severe acute respiratory syndrome coronavirus 2 (SARS-CoV-2), from the affected patients (Zhu, et al., “A Novel Coronavirus from Patients with Pneumonia in China” N Engl J Med (2020) doi: 10.1056/NEJMoa2001017. [Epub ahead of print], Chan, et al., “A familial cluster of pneumonia associated with the 2019 novel coronavirus indicating person-to-person transmission: a study of a family cluster.” Lancet (2020) doi: 10.1016/S0140-6736(20)30154-9 [Epub ahead of print], Zhou, et al., “A pneumonia outbreak associated with a new coronavirus of probable bat origin.” Nature (2020) doi: 10.1038/s41586-020-2012-7 [Epub ahead of print]). This new virus has been recently named as severe acute respiratory syndrome coronavirus 2 (SARS-CoV-2) by the Coronavirus Study Group of the International Committee on Taxonomy of Viruses Most patients with SARS-CoV-2 infection, or Coronavirus Disease 2019 (Covid-19), present with acute onset of fever, myalgia, cough, dyspnea, and radiological evidence of ground-glass lung opacities compatible with atypical pneumonia (Huang, et al., “Clinical features of patients infected with 2019 novel coronavirus in Wuhan, China.” Lancet (2020) doi: 10.1016/S0140-6736(20)30183-5 [Epub ahead of print], Chen, et al., “Epidemiological and clinical characteristics of 99 cases of 2019 novel coronavirus pneumonia in Wuhan, China: a descriptive study.” Lancet (2020) doi: 10.1016/S0140-6736(20)30211-7 [Epub ahead of print], Wang, et al., “Clinical Characteristics of 138 Hospitalized Patients With 2019 Novel Coronavirus-Infected Pneumonia in Wuhan, China.” JAMA (2020) doi: 10.1001/jama.2020.1585. [Epub ahead of print]). However, asymptomatic or mildly symptomatic cases have also been reported (Chan, et al., “A familial cluster of pneumonia associated with the 2019 novel coronavirus indicating person-to-person transmission: a study of a family cluster.” Lancet (2020) doi: 10.1016/S0140-6736(20)30154-9. [Epub ahead of print], Wei, et al., “Novel Coronavirus Infection in Hospitalized Infants Under 1 Year of Age in China.” JAMA (2020) doi: 10.1001/jama.2020.2131. [Epub ahead of print]). Initial epidemiological investigations have indicated the Huanan seafood wholesale market in Wuhan as a geographically linked source, but subsequent detailed epidemiological assessment has revealed that up to 45% of the early cases with symptom onset before 1 Jan. 2020 were not linked to this market (Huang, et al., “Clinical features of patients infected with 2019 novel coronavirus in Wuhan, China.” Lancet (2020) doi: 10.1016/S0140-6736(20)30183-5. [Epub ahead of print], Li, et al., “Early Transmission Dynamics in Wuhan, China, of Novel Coronavirus-Infected Pneumonia.” N Engl J Med (2020) doi: 10.1056/NEJMoa2001316. [Epub ahead of print]). Person-to-person transmissions among close family contacts and healthcare workers, including those without travel history to Wuhan, have been reported (Chan, et al., “A familial cluster of pneumonia associated with the 2019 novel coronavirus indicating person-to-person transmission: a study of a family cluster.” Lancet (2020) doi: 10.1016/S0140-6736(20)30154-9. [Epub ahead of print], Wang, et al., “Clinical Characteristics of 138 Hospitalized Patients With 2019 Novel Coronavirus-Infected Pneumonia in Wuhan, China.” JAMA (2020) doi: 10.1001/jama.2020.1585. [Epub ahead of print], Phan, et al., “Importation and Human-to-Human Transmission of a Novel Coronavirus in Vietnam.” N Engl J Med (2020) doi: 10.1056/NEJMc2001272. [Epub ahead of print], Khan, et al., “Novel coronavirus: how the things are in Wuhan.” Clin Microbiol Infect (2020) doi: 10.1016/j.cmi.2020.02.005.)). Therefore, clinical features and epidemiological links to Wuhan alone are not reliable for establishing the diagnosis of Covid-19.

As evidenced by previous epidemics caused by SARS-CoV and Middle East respiratory syndrome coronavirus (MERS-CoV), highly sensitive and specific laboratory diagnostics for Covid-19 are essential for case identification, contact tracing, animal source finding, and rationalization of infection control measures (Peiris, et al., “Coronavirus as a possible cause of severe acute respiratory syndrome.” Lancet (2003) 361:1319-1325, Cheng, et al., “Severe acute respiratory syndrome coronavirus as an agent of emerging and reemerging infection.” Clin Microbiol Rev (2007) 20:660-694, Chan, et al., “Middle East respiratory syndrome coronavirus: another zoonotic betacoronavirus causing SARS-like disease.” Clin Microbiol Rev (2015) 28:465-522)). The use of viral culture for establishing acute diagnosis is not practical as it takes at least three days for SARS-CoV-2 to cause clear cytopathic effects in selected cell lines, such as Vero, VeroE6 and Huh-7 cells (Zhou, et al., “A pneumonia outbreak associated with a new coronavirus of probable bat origin.” Nature (2020) doi: 10.1038/s41586-020-2012-7. [Epub ahead of print]). Moreover, isolation of the virus requires biosafety level-3 facilities which are not available in most healthcare institutions. Serum antibody and antigen detection tests have not yet been validated, and there may be cross-reactivity with SARS-CoV which shares a high degree (˜82%) of nucleotide identity with SARS-CoV-2 (Chan, et al., “Genomic characterization of the 2019 novel human-pathogenic coronavirus isolated from a patient with atypical pneumonia after visiting Wuhan.” Emerg Microbes Infect (2020) 9:221-236)). A protocol of the first real-time RT-PCR assays targeting the RNA-dependent RNA polymerase (RdRp), envelope (E), and nucleocapsid (N) genes of SARS-CoV-2 were published on 23 Jan. 2020 (Corman, et al., “Detection of 2019 novel coronavirus (2019-nCoV) by real-time RT-PCR.” Euro Surveill (2020) doi: 10.2807/1560-7917.ES.2020.25.3.2000045)). Notably, these assays were designed and validated using synthetic nucleic acid technology and in the absence of available SARS-CoV-2 isolates or original patient specimens (Corman, et al., “Detection of 2019 novel coronavirus (2019-nCoV) by real-time RT-PCR.” Euro Surveill (2020) doi: 10.2807/1560-7917.ES.2020.25.3.2000045)). The reported RdRp assays had been implemented in >30 laboratories in Europe (Reusken, et al., “Laboratory readiness and response for novel coronavirus (2019-nCoV) in expert laboratories in 30 EU/EEA countries, January 2020.” Euro Surveill (2020) doi: 10.2807/1560-7917.ES.2020.25.6.2000082. [Epub ahead of print]). Highly sensitive and specific laboratory diagnostics are important for controlling the rapidly evolving SARS-CoV-2-associated Coronavirus Disease 2019 (Covid-19) epidemic because such assays would be useful in detecting low viral loads and accordingly, identify even asymptomatic subjects.

It is an object of the present invention to provide compositions, methods, and kits for detecting and diagnosing SARS-CoV-2.

It is a further object of the present invention to provide compositions, methods, and kits for detecting and diagnosing SARS-CoV-2which show improved sensitivity and specificity relative to existing detection methods.

### SUMMARY OF THE INVENTION

Sequences for detection of SARS-CoV-2 are provided and can, for example, include or consist of a sequence of any of SEQ ID NOS:1-12, a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s), or a combination thereof relative thereto, or the reverse complement of any of the foregoing.

The sequences can be targeted by probes and primers. Thus, probes and primers that target the foregoing target sequences and methods of use thereof for the detection and diagnosis of SARS-CoV-2 are also provided. In some embodiments, the primers or probes hybridizes with a sequence of any of SEQ ID NOS:1-12, a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s), or a combination thereof relative thereto, or the reverse complement of any of the foregoing.

Particularly preferred primer sets and probes are provided in Table 1 and can be used alone or in combination. In some embodiments, the probes are modified to include a detectable reporter such as a radioactive or fluorescent label. In particularly embodiments, the probe are modified for use a realtime polymerase chain reaction, and include, for example, one or more fluorescent reporters, one or more quenchers, or a combination thereof. In a non-limiting example, the probes exemplified in Table 1 include a 5′ fluorescent reporter and a 3′ quencher.

The target sequences, primers, and probes can use in methods of detecting SARS-CoV-2 nucleic acids in a sample such as mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood, thus, methods of detecting SARS-CoV-2 in such samples are also provided. The sample can be one that is isolated from a subject that may have been exposed to or is suspected of having SARS-CoV-2. In some embodiments, the sample is processed to expose or isolate nucleic acids from sample before it is subjected to the detection method.

Detection methods include, but are not limited to, microarray, differential display, RNase protection assay, northern blot, reverse transcriptase (RT) polymerase chain reaction (PCR), and combinations thereof. In preferred embodiments, the detection methods include RT-PCT, more preferably realtime or quantitative RT-PCR, most preferably wherein the RT-PCR includes target specific reverse transcription and/or target specific PCR. In preferred embodiments, the methods can be used to detect one or more of the sequences for detection disclosed herein, including, but not limited to any one of SEQ ID NOS:1-12, and the reverse complements thereof. Preferred primer sets for reverse transcription and/or PCR include: SEQ ID NOS:1 and 2, which can optionally be used in combination with a probe of SEQ ID NO:3; SEQ ID NOS:5 and 6, which can optionally be used in combination with a probe of SEQ ID NO:7; and SEQ ID NOS:9 and 10, which can optionally be used in combination with a probe of SEQ ID NO:11. Any of the foregoing sets or primers and optionally probes can be used in combinations of 2 or even 3 for multiplex reactions. Detection can include identification of one or more amplicons formed by PCR utilizing one or more of the primer pairs, optionally via detection of fluorescence from the probe.

In some embodiments, the disclosed primers, probes, compositions, or methods are more sensitive, selective, or combination thereof for SARS-CoV-2 relative to one or more other human- and/or non-human pathogenic coronaviruses and/or respiratory pathogens, such as SARS-CoV, MERS-CoV, HCoV-OC43, HCoV-229E, HCoV-NL63, adenovirus, human metapneumovirus, influenza A (H1N1 and H3N2) viruses, influenza B virus, influenza C virus, parainfluenza viruses types 1 to 4, rhinovirus, respiratory syncytial virus, Bat-SL-CoV, and combinations thereof. Thus, in some embodiments, one or more non-SARS-CoV-2 virus cannot be detected according the disclosed compositions or methods. In particular embodiments, the undetectable non-SARS-CoV-2 virus is SARS-CoV and/or Bat-SL-CoV.

Methods of diagnosing a subject with SARS-CoV-2 are also provided and can include analyzing a sample from the subject according to a detection method, wherein detection of SARS-CoV-2 in the sample indicates the subject has SARS-CoV-2.

### BRIEF DESCRIPTION OF THE DRAWINGS

FIGS. 1A-1D are partial alignments of the oligonucleotide regions of SARS-CoV-2 (2019-nCoV HKU-SZ-002a 2020, GenBank accession no. MN938384; and 2019-nCoV HKU-SZ-005b 2020, GenBank accession no. MN975262), human SARS-CoV (SARS-CoV HKU-39849, GenBank accession no. AY278491; and SARS-CoV GZ50, GenBank accession no. AY304495), and bat SARS-like coronaviruses (Bat-SL-CoVZC45, GenBank accession no. MG772933; and Bat-SL-CoVZXC21, GenBank accession no. MG772934) predicted to bind with the new (1A) Covid-19-RdRp/Hel, (1B) Covid-19-S, (1C) Covid-19-N, and the published (1D) RdRp-P2 real-time RT-PCR assays.

### DETAILED DESCRIPTION OF THE INVENTION

### I. Definitions

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3” is complementary to the sequence “3′-T-C-A-S′.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The term “substantially complementary” as used herein means that two sequences hybridize. In some embodiments, the hybridization occurs only under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (T.sub.m) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “primer pair” refers to a forward and reverse primer pair (i.e., a left and right primer pair) that can be used together to amplify a given region of a nucleic acid of interest.

“Probe” as used herein refers to a nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. Probes are typically at least about 10, 15, 20, 25, 30, 35, 40, 50, 60, 75, 100 nucleotides or more in length.

As used herein, the term “sample” refers to in vitro as well as clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material), nasopharyngeal aspirate, nasopharyngeal swab, throat swab, and other discussed herein and otherwise known in the art.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 85-95%, and more preferably at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

“Sensitivity” as used herein, is a measure of ability of a detection assay to directly or indirectly detect the presence of a target sequence (e.g., a SARS-CoV-2 viral sequence) in a sample.

“Specificity,” as used herein, is a measure of the ability of a detection assay to distinguish a truly occurring target sequence (e.g., a SARS-CoV-2 viral sequence) from other closely related sequences (e.g., other human-pathogenic coronaviruses and respiratory pathogens). It is the ability to avoid false positive detections.

The term “stringent hybridization conditions” as used herein refers to hybridization conditions at least as stringent as the following: hybridization in 50% formamide, 5×SSC, 50 mM NaH2PO4, pH 6.8, 0.5% SDS, 0.1 mg/mL sonicated salmon sperm DNA, and 5× Denhart's solution at 42 C. overnight; washing with 2×SSC, 0.1% SDS at 45 C.; and washing with 0.2×SSC, 0.1% SDS at 45 C. In another example, stringent hybridization conditions should not allow for hybridization of two nucleic acids which differ over a stretch of 20 contiguous nucleotides by more than two bases.

The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### II. Compositions

A. Target, Probe, and Primer Sequences

Primers and probes for use in the detection of RNA-dependent RNA polymerase (RdRp)/helicase (Hel), spike (S), and nucleocapsid (N) genes of SARS-CoV-2 are provided. Although sometime referred to herein is a probe or a primer, it will be appreciated that any of the probe sequences and the reverse complements thereof can be used as primer sequences, and any of the primer sequences and the reverse complements thereof can be used as probe sequences. All of the probes are expressly provided with and without detection labels (e.g., fluorophores, etc.)

The positive-sense, single-stranded RNA genome of SARS-CoV-2 is ˜30 kilobases in size and encodes ˜9860 amino acids. The disclosed probes and primers are typically designed to hybridize with a target SARS-CoV-2 genomic sequence, or the reverse complement thereof that can be generated by reverse transcription. In some embodiments, the SARS-CoV-2 genomic sequence is the sequence of GenBank accession no. MN975262.

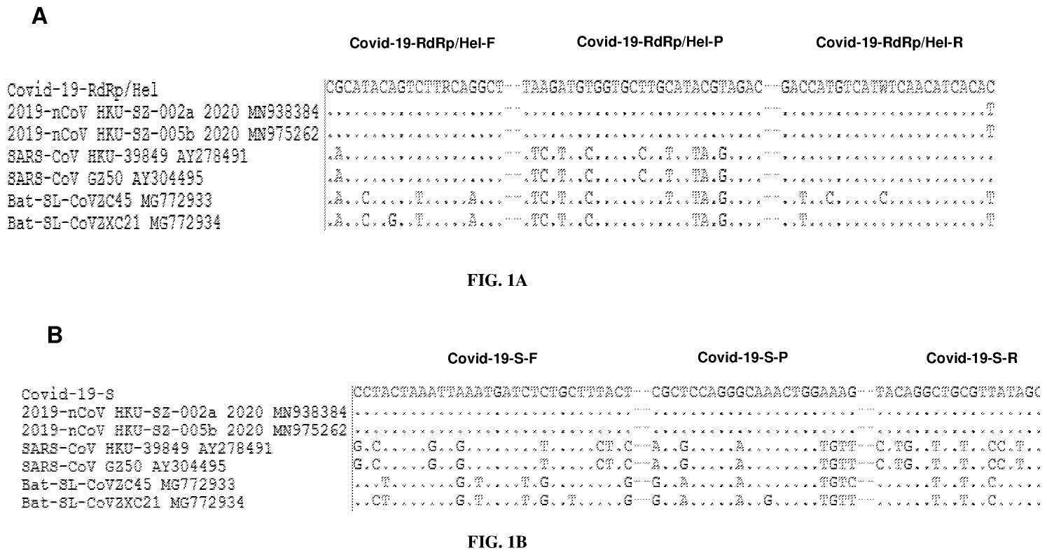

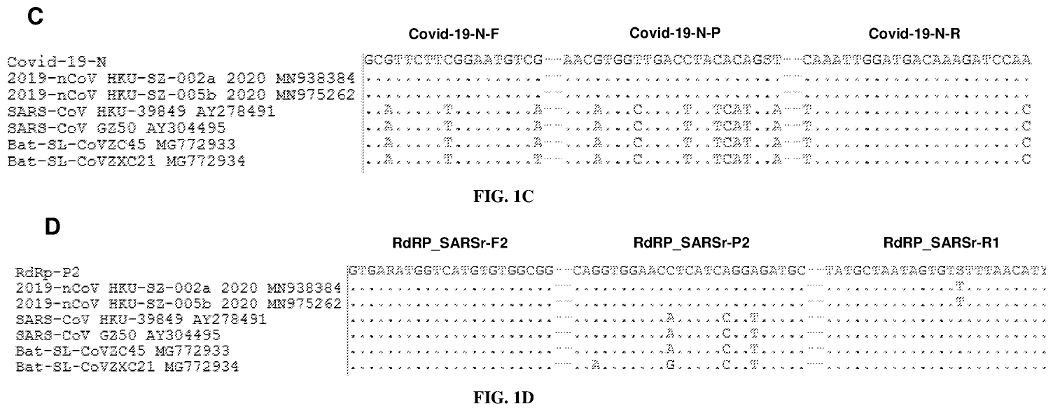

The target nucleic acid to which the probes and/or primer hybridize can be single stranded or double stranded RNA or DNA (e.g., single stranded or duplex cDNA), and can be genomic (positive strand) sequence, or the reverse complement thereof. In some embodiments, the probe(s) and/or primer(s) are can detect and/or facilitate transcription, strand synthesis, and/or amplification of a target gene selected from SARS-CoV-2 RdRp/Helicase, Spike, or Nucleocapsid. Suitable target sequences are outlined in FIGS. 1A-1D and discussed in more detail below.

In some embodiments, the probe or primer is substantially complementary or complementary to the target genomic sequence or reverse complement thereof. In some embodiments, the probe or primer hybridizes to the target genomic sequence or reverse complement thereof under stringent hybridization conditions.

In some embodiments, the probes and primers do not hybridize, do not hybridize under stringent conditions, are unsuitable for detection, transcription, strand synthesis, and/or amplification of the corresponding or homologous target genomic sequence, or any combination thereof, of one or more other human- and/or non-human pathogenic coronaviruses or respiratory pathogens, including, but not limited to, SARS-CoV, MERS-CoV, HCoV-OC43, HCoV-229E, HCoV-NL63, adenovirus, human metapneumovirus, influenza A (H1N1 and H3N2) viruses, influenza B virus, influenza C virus, parainfluenza viruses types 1 to 4, rhinovirus, and respiratory syncytial virus, Bat-SL-CoV, etc., and others discussed herein and illustrated in FIGS. 1A-1D, and otherwise known in the art.

For the sequences herein, N denotes A, G, T/U, or C; R denotes A or G; Y denotes T/U or C; K denotes G or T/U; and W denotes A or T/U; and S denotes C or G. For all DNA sequences herein, the corresponding RNA sequence is also expressly provided. For all nucleic acid sequences provided herein, the corresponding complementary sequence and reverse complementary sequence are also expressly provided.

1. (RdRp)/Helicase (Hel)

Sequences useful for targeting RdRp/helicase (Hel) for detection, transcription, strand synthesis, and amplification include those comprising or consisting of CGCATACAGTCTTRCAGGCT (SEQ ID NO:1), GTGTGATGTTGAWATGACATGGTC (SEQ ID NO:2) TTAAGATGTGGTGCTTGCATACGTAGAC (SEQ ID NO:3), GACCATGTCATWTCAACATCACAC (SEQ ID NO:4), the reverse complement of any of SEQ ID NOS:1-4, or a nucleic acid sequence having at least 70%, 75%, 80%, 85%, 90%, 95%, 96%, 97%, 98%, or 99% sequence identity to any of the foregoing.

In some embodiments, the probe or primer is designed to hybridize to a single or double stranded genomic target sequence including or consisting of any one or more of SEQ ID NOS:1-4 or the reverse complement thereof, or a variant thereof having at least 70%, 75%, 80%, 85%, 90%, 95%, 96%, 97%, 98%, or 99% sequence identity thereto. Thus, in some embodiments, the probe or primer hybridizes, hybridizes under stringent conditions, is suitable for detection, transcription, strand synthesis, and/or amplification, or any combination thereof, of a genomic target sequence with up to 1, 2, 3, 4, 5, 6, 7, 8, 9, 10, or more nucleic acid substitution(s), addition(s), deletion(s), or a combination thereof relative to any one SEQ ID NO:1-4, or the reverse complement thereof. In some embodiments, the probe or primer does not hybridize, does not hybridize under stringent conditions, is unsuitable for detection, transcription, strand synthesis, and/or amplification of the target genomic sequence, or any combination thereof, with a genomic target sequence with 10, 9, 8, 7, 6, 5, 4, 3, 2, or 1, nucleic acid substitution(s), addition(s), deletion(s), or a combination thereof relative to any one of SEQ ID NOS:1-4, or the reverse complement thereof.

In some embodiments a primer or probe comprises or consists of CGCATACAGTCTTRCAGGCT (SEQ ID NO:1), GTGTGATGTTGAWATGACATGGTC (SEQ ID NO:2), TTAAGATGTGGTGCTTGCATACGTAGAC (SEQ ID NO:3), or the reverse complement thereof, or a variant of any of the foregoing having at least 70%, 75%, 80%, 85%, 90%, 95%, 96%, 97%, 98%, or 99% sequence identity thereto.

In some embodiments, the target sequence, probe, or primer of SEQ ID NO:3 lacks the 5′ “T”. Thus, SEQ ID NO:3 lacking the 5′ “T” (e.g., TAAGATGTGGTGCTTGCATACGTAGAC (SEQ ID NO:13)) and its reverse complement are also expressly disclosed, can be used herein in addition to, or alternative to, SEQ ID NO:3. Thus, all embodiments disclosed with respected to SEQ ID NO:3 are also expressly disclosed with, and can be substituted by, SEQ ID NO:13.

2. Spike (S)

Sequences useful for targeting Spike (S) for detection, transcription, strand synthesis, and amplification include those comprising or consisting of CCTACTAAATTAAATGATCTCTGCTTTACT (SEQ ID NO:5), CAAGCTATAACGCAGCCTGTA (SEQ ID NO:6), CGCTCCAGGGCAAACTGGAAAG (SEQ ID NO:7), TACAGGCTGCGTTATAGCTTG (SEQ ID NO:8), the reverse complement of any of SEQ ID NOS:5-8, or a nucleic acid sequence having at least 70%, 75%, 80%, 85%, 90%, 95%, 96%, 97%, 98%, or 99% sequence identity to any of the foregoing.

In some embodiments, the probe or primer is designed to hybridize to a single or double stranded genomic target sequence including or consisting of any one or more of SEQ ID NOS:5-8 or the reverse complement thereof, or a variant thereof having at least 70%, 75%, 80%, 85%, 90%, 95%, 96%, 97%, 98%, or 99% sequence identity thereto. Thus, in some embodiments, the probe or primer hybridizes, hybridizes under stringent conditions, is suitable for detection, transcription, strand synthesis, and/or amplification, or any combination thereof, of a genomic target sequence with up to 1, 2, 3, 4, 5, 6, 7, 8, 9, 10, or more nucleic acid substitution(s), addition(s), deletion(s), or a combination thereof relative to any one SEQ ID NO:5-8, or the reverse complement thereof. In some embodiments, the probe or primer does not hybridize, does not hybridize under stringent conditions, is unsuitable for detection, transcription, strand synthesis, and/or amplification of the target genomic sequence, or any combination thereof, with a genomic target sequence with 10, 9, 8, 7, 6, 5, 4, 3, 2, or 1, nucleic acid substitution(s), addition(s), deletion(s), or a combination thereof relative to any one of SEQ ID NOS:5-8, or the reverse complement thereof.

In some embodiments a primer or probe comprises or consists of CCTACTAAATTAAATGATCTCTGCTTTACT (SEQ ID NO:5), CAAGCTATAACGCAGCCTGTA (SEQ ID NO:6), CGCTCCAGGGCAAACTGGAAAG (SEQ ID NO:7), or the reverse complement thereof, or a variant of any of the foregoing having at least 70%, 75%, 80%, 85%, 90%, 95%, 96%, 97%, 98%, or 99% sequence identity thereto.

3. Nucleocapsid (N)

Sequences useful for targeting Nucleocapsid (N) for detection, transcription, strand synthesis, and amplification include those comprising or consisting of GCGTTCTTCGGAATGTCG (SEQ ID NO:9), TTGGATCTTTGTCATCCAATTTG (SEQ ID NO:10), AACGTGGTTGACCTACACAGST (SEQ ID NO:11), CAAATTGGATGACAAAGATCCAA (SEQ ID NO:12), the reverse complement of any of SEQ ID NOS:9-12, or a nucleic acid sequence having at least 70%, 75%, 80%, 85%, 90%, 95%, 96%, 97%, 98%, or 99% sequence identity to any of the foregoing.

In some embodiments, the probe or primer is designed to hybridize to a single or double stranded genomic target sequence including or consisting of any one or more of SEQ ID NOS:9-12 or the reverse complement thereof, or a variant thereof having at least 70%, 75%, 80%, 85%, 90%, 95%, 96%, 97%, 98%, or 99% sequence identity thereto. Thus, in some embodiments, the probe or primer hybridizes, hybridizes under stringent conditions, is suitable for detection, transcription, strand synthesis, and/or amplification, or any combination thereof, of a genomic target sequence with up to 1, 2, 3, 4, 5, 6, 7, 8, 9, 10, or more nucleic acid substitution(s), addition(s), deletion(s), or a combination thereof relative to any one SEQ ID NO:9-12, or the reverse complement thereof. In some embodiments, the probe or primer does not hybridize, does not hybridize under stringent conditions, is unsuitable for detection, transcription, strand synthesis, and/or amplification of the target genomic sequence, or any combination thereof, with a genomic target sequence with 10, 9, 8, 7, 6, 5, 4, 3, 2, or 1, nucleic acid substitution(s), addition(s), deletion(s), or a combination thereof relative to any one of SEQ ID NOS:9-12, or the reverse complement thereof.

In some embodiments a primer or probe comprises or consists of GCGTTCTTCGGAATGTCG (SEQ ID NO:9), TTGGATCTTTGTCATCCAATTTG (SEQ ID NO:10), AACGTGGTTGACCTACACAGST (SEQ ID NO:11), or the reverse complement thereof, or a variant of any of the foregoing having at least 70%, 75%, 80%, 85%, 90%, 95%, 96%, 97%, 98%, or 99% sequence identity thereto.

4. Exemplary Probes and Primers

Exemplary targets, primers and probes are provided below in Table 1 and FIGS. 1A-1D.

TABLE 1
Exemplary primer and probe sequences

| | | Genome | Primer/ | |
|---|---|---|---|---|
| Assay | Gene target | location^{a} | probe | Sequence (5′ to 3′) |
| Covid-19- | RdRp/Helicase | 16220-16239 | Forward | CGCATACAGTCTTRCAGGCT |
| RdRp/Hel | | | | (SEQ ID NO: 1) |
| | | 16330-16353 | Reverse | GTGTGATGTTGAWATGACATGGTC |
| | | | | (SEQ ID NO: 2) |
| | | 16276-16303 | Probe | FAM-TTAAGATGTGGTGCTTGC |
| | | | | ATACGTAGAC-1ABKFQ |
| | | | | (SEQ ID NO: 3) |
| Covid-19-S | Spike | 22712-22741 | Forward | CCTACTAAATTAAATGATCTCTGCTTTACT |
| | | | | (SEQ ID NO: 5) |
| | | 22849-22869 | Reverse | CAAGCTATAACGCAGCCTGTA |
| | | | | (SEQ ID NO: 6) |
| | | 22792-22813 | Probe | HEX-CGCTCCAGGGCAAACTGGAAAG-IABkFQ |
| | | | | (SEQ ID NO: 7) |
| Covid-19-N | Nucleocapsid | 29210-29227 | Forward | GCGTTCTTCGGAATGTCG |
| | | | | (SEQ ID NO: 9) |
| | | 29284-29306 | Reverse | TTGGATCTTTGTCATCCAATTTG |
| | | | | (SEQ ID NO: 10) |
| | | 29257-29278 | Probe | FAM-AACGTGGTTGACCTACACAGST-1ABKFQ |
| | | | | (SEQ ID NO: 11) |

^{a}The genome sequence of SARS-COV-2 (strain HKU-SZ-005b 2020) was used as reference (GenBank accession no. MN975262).
Abbreviations: RdRp, RNA-dependent RNA polymerase; FAM, 6-carboxyfluorescein

B. Modifications to Probes and Primers

Any of the probes and primers can include one or more modifications to enhance, improve or facilitate its desired function. Common modifications include those that enhance detection including radioactive labels (radiolabels), fluorescent reporters (e.g., fluorophores and/or quenchers), attachment moieties (e.g., amine, glycerol, phosphate, thiol, etc.), binding moieties (e.g., biotin, digoxigenin, dinitrophenol, etc.), and/or antisense enhancers, or are spacers, analogs, intercalation agents, or phosphorothioates. Modifications can be used in any way suitable for performing the desired function. For example, modifications can be made at the 3′ end, the 5′ end, internally, or any combination thereof of the primer or probe.

Fluorophore and quencher modifications, particularly those suitable for detection during realtime PCR, are particularly advantageous for the disclosed compositions, particularly probes. Single-quenched and double-quenched probes are contemplated. Double-quenched probes may provide consistently lower background, resulting in higher signal compared to single-quenched probes. Double-quenched probes may include, e.g., ZEN™ or TAO™ molecules as a secondary, internal quencher allowing for longer probe lengths to be used in addition to providing strong quenching and increased signal.

Exemplary fluorescent modifications include, but are not limited to, 6-FAM™ (fluorescein), ROX™, Cyanine 3, Cyanine 5, Cyanine 5.5, 6-FAM dT, HEX™, JOE™, 6-Carboxy-rhodamine 6G™, TAMRA, TAMRA NHS Ester, TET™, TxRd, (Sulforhodamine 101-X), A488 (Sulfonated Fluorescein 488), WellRED D2-PA, WellRED D3-PA, and WellRED D4-PA.

Traditional dark quenchers that absorb broadly and do not emit light, which allows use of multiple reporter dyes with the same quencher. This characteristic allows for expanded options for multiplex assays. Dark quenchers reduce signal cross-talk, simplifying reporter dye detection, making them compatible with a broad range of image analysis instruments. Examples of dark quenchers include Black Hole Quenchers, and Iowa Black FQ and RQ, and the internal ZEN Quencher. Other suitable quenchers include, but are not limited to, BHQ1 and IABkFQ.

The probes in Table 1 are exemplified with 5′ FAM or HEX fluroscent reporters and 3′ lABkFQ quenchers.

### III. Methods of Use

The disclosed target sequences, probes, and primers can be used in methods of detecting SARS-CoV-2. Methods typically involve directly or indirectly detecting virus (e.g., viral genome) in a biological sample.

A. Samples and Sample Preparation

Biological samples include, but are not limited to, tissue or bodily fluid collected from a subject having or suspected of having the virus. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Preferred sample sources include mucus, rectal swab, nasopharyngeal aspirate, nasopharyngeal swab, throat swab, feces, sputum, plasma, serum, or whole blood. In some embodiments, the sample is treated with a DNAase.

The methods may include sample preparation. Sample preparation methods for detection of virus in biological samples are known in the art, and exemplified below. For example, in some embodiments, total nucleic acid (TNA) extraction of clinical specimens and laboratory cell culture of viral isolates are performed using a kit such as NucliSENS easyMAG extraction system. The volume of the specimens used for extraction and the elution volume depended on the specimen type and the available amount of the specimen. In the Examples below, in general, 250 μl of each respiratory tract specimen, urine, rectal swab, and feces were subjected to extraction with an elution volume of 55 μl and 1000 μl of each plasma specimen were subjected to extraction with an elution volume of 25 μl.

B. Methods of Detection

Methods of using the disclosed primers and probes to detect virus in a sample are known and include, for example, microarrays, differential display, RNase protection assays, northern blot, and RT-PCR.

In preferred embodiments, the method of detection is reverse transcriptase (RT) polymerase chain reaction (PCR), preferably target sequence-specific RT-PCR, most preferably, target sequence-specific quantitative or realtime RT-PCR. RT-PCR is a variant of polymerase chain reaction (PCR), a laboratory technique commonly used in molecular biology to generate many copies of a deoxyribonucleic acid (DNA) sequence, a process termed “amplification.” In RT-PCR, a ribonucleic acid (RNA) strand is first reverse transcribed into its DNA complement (complementary DNA, or cDNA) using the enzyme reverse transcriptase. The resulting cDNA is subsequently amplified using traditional PCR. RT-PCR utilizes a pair of primers, which are complementary to a defined sequence on each of the two strands of the cDNA. These primers are then extended by a DNA polymerase and a copy of the strand is made after each PCR cycle, leading to exponential amplification. It has been discovered that the disclosed compositions, methods and kits provide alternate gene regions of the SARS-CoV-2 to target in an RT-PCR assay, as well as design of probes used in RT-PCR, that result in an RT-PCR method for detecting SARS-CoV-2 that has improved sensitivity and specificity over alternative methods.

A reverse transcription (RT) reaction refers to the process in which single-stranded RNA is reverse transcribed into complementary DNA (cDNA) by using total cellular RNA or poly(A) RNA, a reverse transcriptase enzyme, one or more primers, dNTPs (refers to a mixture of equal molar of dATP, dTTP, dCTP, and dGTP), and typically an RNase inhibitor. Primers for an RT reaction can be random primers (e.g., random hexamers) or oligo dT for cDNA production from total RNA or polyA RNA (mRNA) respectively, or can be sequence-specific to drive selective cDNA preparation of only a target sequence or sequence(s). The disclosed methods typically include sequence specific RT primer(s). Exemplary primers those disclosed above.

General methods and kits including reaction components for reverse transcription are known and the art and can be employed in the disclosed methods.

A typical reaction mixture includes RNA, primer, dNTP nucleotide mixture, reverse transcriptase, RNase inhibitor, buffer including Tris-HCl, KCl, MgCl₂, DTT, and nuclease free water up to the desired reaction volume.

Next, PCR can be used for second strand synthesis (e.g., to form double stranded cDNA amplicons), and for amplification of the cDNA template. PCR typically relies on a forward and reverse primer (e.g., a primer set). Preferably, the forward and reverse primers specifically amplify the target region whose detection or quantification is desired. In some embodiments, at least one of the PCR primers is the same as at least one of the RT primers.

Other reagents for second strand synthesis and PCR can include, but are not limited to, a DNA polymerase (e.g., heat-resistant Taq polymerase), dNTPs, a buffer solution providing a suitable chemical environment for optimum activity and stability of the DNA polymerase, bivalent cations, typically magnesium (Mg) or manganese (Mn) ions, etc.

The RT reaction and PCR cycle(s) can be carried out as separate and distinct reactions, or in a single tube using a thermocycler as is known in the art and exemplified below.

In preferred embodiments, the RT-PCR is quantitative or realtime PCR. Such assays include non-specific detection: real-time PCR with double-stranded DNA-binding dyes as reporters, where a DNA-binding dye binds to all double-stranded (ds) DNA in PCR, increasing the fluorescence quantum yield of the dye. An increase in DNA product during PCR therefore leads to an increase in fluorescence intensity measured at each cycle. However, dsDNA dyes such as SYBR Green will bind to all dsDNA PCR products, including nonspecific PCR products (such as Primer dimer). This can potentially interfere with, or prevent, accurate monitoring of the intended target sequence.

In real-time PCR with dsDNA dyes the reaction is prepared as usual, with the addition of fluorescent dsDNA dye. Then the reaction is run in a real-time PCR instrument, and after each cycle, the intensity of fluorescence is measured with a detector; the dye only fluoresces when bound to the dsDNA (i.e., the PCR product). This method has the advantage of only needing a pair of primers to carry out the amplification, which keeps costs down; multiple target sequences can be monitored in a tube by using different types of dyes.

In preferred embodiments, the detect assay is specific detection by realtime RT-PCR carried out using a fluorescent reporter probe. Florescent reporter probes detect only the DNA containing the sequence complementary to the probe; therefore, use of the reporter probe significantly increases specificity, and enables performing the technique even in the presence of other dsDNA. Using different-colored labels, fluorescent probes can be used in multiplex assays for monitoring several target sequences in the same tube. The specificity of fluorescent reporter probes also prevents interference of measurements caused by primer dimers, which are undesirable potential by-products in PCR.

The method relies on a DNA-based probe with a fluorescent reporter at one end and a quencher of fluorescence at the opposite end of the probe. Suitable probe sequences, and well as exemplary fluorescent reports and quenchers are discussed above.

The close proximity of the reporter to the quencher prevents detection of its fluorescence; breakdown of the probe by the 5′ to 3′ exonuclease activity of the (e.g., Taq) polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter.

The RT-PCR is prepared as is known in the art and exemplified below, and the reporter probe is added. As the reaction commences, during the annealing stage of the PCR both probe and primers anneal to the DNA target. Polymerization of a new DNA strand is initiated from the primers, and once the polymerase reaches the probe, its 5′-3′-exonuclease degrades the probe, physically separating the fluorescent reporter from the quencher, resulting in an increase in fluorescence.

Fluorescence is detected and measured in a real-time PCR machine, and its geometric increase corresponding to exponential increase of the product is used to determine the quantification cycle (Cq) in each reaction.

Real-time RT-PCR assays for SARS-CoV-2 RNA detection were exemplified below using QuantiNova Probe RT-PCR Kit (Qiagen) in a LightCycler 480 Real-Time PCR System (Roche, Basel, Switzerland) as previously described (Chan, et al., “Improved detection of Zika virus RNA in human and animal specimens by a novel, highly sensitive and specific real-time RT-PCR assay targeting the 5′-untranslated region of Zika virus.” Trop Med Int Health (2017) 22:594-603)). Each reaction mixture contained QuantiNova Probe RT-PCR Master Mix, QN Probe RT-Mix, forward and reverse primer, probe, 1.2 μl TNA as the template.

In some embodiments, the assays are multiplexed to detect two or more target sequences (e.g., RdRp/helicase (Hel), Spike (S), and/or Nucleocapsid (N)), at once.

In preferred embodiments, the disclosed methods are sensitive and/or specific for detection of SARS-CoV-2 in a sample. Preferable, positive detection of SAR-CoV-2 is accompanied by the absence of detection of (i.e., negative for), other human- and/or non-human pathogenic coronaviruses or respiratory pathogens, including, but not limited to, SARS-CoV, MERS-CoV, HCoV-OC43, HCoV-229E, HCoV-NL63, adenovirus, human metapneumovirus, influenza A (H1N1 and H3N2) viruses, influenza B virus, influenza C virus, parainfluenza viruses types 1 to 4, rhinovirus, and respiratory syncytial virus, Bat-SL-CoV, etc., and others discussed herein and illustrated in FIGS. 1A-1D, and otherwise known in the art. In preferred embodiments, the methods are more sensitive and/or specific for SARS-CoV-2 than RdRp-P2 assay and/or show lower cross-reactivity with SARS-CoV relative thereto. The RdRp-P2 assay is described in (Corman, et al., “Detection of 2019 novel coronavirus (2019-nCoV) by real-time RT-PCR.” Euro Surveill (2020) doi: 10.2807/1560-7917.ES.2020.25.3.2000045)).

Exemplary assays were carried out as discussed in the examples below, using the primers and probes provided in Table 1.

Among the three assays tested, the Covid-19-RdRp/Hel assay (e.g., SEQ ID NOS:1-3) had the lowest limit of detection in vitro (1.8 TCID50/ml with genomic RNA and 11.2 RNA copies/reaction with in vitro RNA transcripts). Among 273 specimens from 15 patients with laboratory-confirmed Covid-19, 77 (28.2%) were positive by both the Covid-19-RdRp/Hel and RdRp-P2 assays. The Covid-19-RdRp/Hel assay was positive for an additional 44 RdRd-P2-negative specimens [119/273 (43.6%) vs 77/273 (28.2%), P<0.001], including 29/120 (24.2%) respiratory tract specimens and 13/153 (8.5%) non-respiratory tract specimens. The mean viral load of these specimens was 3.21×10⁴ RNA copies/ml (range, 2.21×10² to 4.71×10⁵ RNA copies/ml). The Covid-19-RdRp/Hel assay did not cross-react with other human-pathogenic coronaviruses and respiratory pathogens in cell culture and clinical specimens, whereas the RdRp-P2 assay cross-reacted with SARS-CoV in cell culture.

In preferred embodiments, the method includes an RdRp/Hel detection assay using one or more of primers and probes described above, preferably a RdRp/Hel-specific quantitative or realtime RT-PCR assay, most particularly utilizing the primers and probe of SEQ ID NOS:1-3.

C. Diagnostic Methods

Diagnostic methods are also provided and can include subjecting a biological sample obtained from the subject (or e.g., total nucleic acid or RNA prepared therefrom) to a SARS-CoV-2 detection method described herein and diagnosing the subject as having SARS-CoV-2 if SARS-CoV-2 (e.g., the target gene such as SARS-CoV-2 RdRp/helicase (Hel), Spike (S), and/or Nucleocapsid (N)) is/are detected.

### IV. Kits

Kits for use with the methods disclosed herein are also disclosed. The kits typically include one or more reagents for lysing cells, isolating nucleic acids, particularly RNA, from cell lysate, reverse transcription, second strand synthesis, purifying cDNA, PCR, or any combination thereof.

Reagents can be, for example, buffers, primers, probes, enzymes, dNTPs, carrier RNA, and other active agents and organics that facilitate various steps of the disclosed reactions. The kits can also include instructions for use.

### EXAMPLES

### Materials and Methods

### Viruses and Clinical Specimens

SARS-CoV-2 was isolated from a patient with laboratory-confirmed Covid-19 in Hong Kong (To, et al., “Consistent detection of 2019 novel coronavirus in saliva.” Clin Infect Dis (2020) doi: 10.1093/cid/ciaa149. [Epub ahead of print]). The viral isolate was amplified by one additional passage in VeroE6 cells to make working stocks of the virus (1.8×10⁷ 50% tissue culture infective doses [TCID₅₀]/ml). For in vitro specificity evaluation, archived laboratory culture isolates (n=17) of other human-pathogenic coronaviruses and respiratory viruses used were obtained from the Department of Microbiology, The University of Hong Kong, as previously described (Chan, et al., “Development and Evaluation of Novel Real-Time Reverse Transcription-PCR Assays with Locked Nucleic Acid Probes Targeting Leader Sequences of Human-Pathogenic Coronaviruses.” J Clin Microbiol (2015) 53:2722-2726)). All experimental protocols involving live SARS-CoV-2, SARS-CoV, and MERS-CoV followed the approved standard operating procedures of the Biosafety Level 3 facility as previously described (Chan, et al., “Differential cell line susceptibility to the emerging novel human betacoronavirus 2c EMC/2012: implications for disease pathogenesis and clinical manifestation.” J Infect Dis (2013) 207:1743-1752, Zhou, et al., “Active replication of Middle East respiratory syndrome coronavirus and aberrant induction of inflammatory cytokines and chemokines in human macrophages: implications for pathogenesis.” J Infect Dis (2014) 209:1331-1342)). For the clinical evaluation study, a total of 273 (120 respiratory tract and 153 non-respiratory tract) clinical specimens were collected from 15 patients with laboratory-confirmed Covid-19 (To, et al., “Consistent detection of 2019 novel coronavirus in saliva.” Clin Infect Dis (2020) doi: 10.1093/cid/ciaa149. [Epub ahead of print]). Additionally, the total nucleic acid extracts of 61 archived (stored at −80° C. until use) FilmArray RP2-tested (22 positive and 39 negative) nasopharyngeal aspirates/swabs and throat swabs collected from 61 adult patients who were managed at hospitals for upper and/or lower respiratory tract symptoms were prepared according to the manufacturer's instructions for assessing potential cross-reactivity of the assays with other respiratory pathogens in clinical specimens. The study was approved by Institutional Review Board of The University of Hong Kong/Hospital Authority (UW 14-249).

### Nucleic Acid Extraction

Total nucleic acid (TNA) extraction of clinical specimens and laboratory cell culture of viral isolates were performed using NucliSENS easyMAG extraction system (BioMerieux, Marcy-l'Étoile, France) according to the manufacturer's instructions and as previously described (Chan, et al., “Differential cell line susceptibility to the emerging novel human betacoronavirus 2c EMC/2012: implications for disease pathogenesis and clinical manifestation.” J Infect Dis (2013) 207:1743-1752)). The volume of the specimens used for extraction and the elution volume depended on the specimen type and the available amount of the specimen. In general, 250 μl of each respiratory tract specimen, urine, rectal swab, and feces were subjected to extraction with an elution volume of 55 μl; and 100 μl of each plasma specimen were subjected to extraction with an elution volume of 25 μl. The extracts were stored at −80° C. until use.

### Primers and Probes

Primer and probe sets targeting different gene regions [RdRp/Helicase (Hel), Spike (S), and N] of SARS-CoV-2 were designed and tested. The probes were predicted to specifically amplify SARS-CoV-2 and had no homologies with human, other human-pathogenic coronaviruses or microbial genes on BLASTn analysis that would potentially produce false-positive test results as previously described (Chan, et al., “Development and Evaluation of Novel Real-Time Reverse Transcription-PCR Assays with Locked Nucleic Acid Probes Targeting Leader Sequences of Human-Pathogenic Coronaviruses.” J Clin Microbiol (2015) 53:2722-2726)). Primer and probe sets with the best amplification performance were selected.

### In Vitro RNA Transcripts for Making Positive Controls and Standards

Linearized pCR2.1-TOPO plasmid (Invitrogen, Carlsbad, CA, USA) with T7 promoter and a cloned target region (RdRp/Hel, S, or N) of SARS-CoV-2 were used for in vitro RNA transcription using MEGAscript T7 Transcription Kit (Ambion, Austin, TX, USA) for the standards and limit of detection as previously described (Chan, et al., “Development and Evaluation of Novel Real-Time Reverse Transcription-PCR Assays with Locked Nucleic Acid Probes Targeting Leader Sequences of Human-Pathogenic Coronaviruses.” J Clin Microbiol (2015) 53:2722-2726, Chan, et al., “Improved detection of Zika virus RNA in human and animal specimens by a novel, highly sensitive and specific real-time RT-PCR assay targeting the 5′-untranslated region of Zika virus.” Trop Med Int Health (2017) 22:594-603)). Each linearized plasmid template was mixed with 2 μl each of ATP, GTP, CTP, and UTP, 10×reaction buffer, and enzyme mix in a standard 20 μl reaction mixture. The reaction mixture was incubated at 37° C. for 16 h, followed by addition of 1 μl of TURBO DNase, and was further incubated at 37° C. for 15 min. The synthesized RNA was cleaned by RNeasy Mini Kit (Qiagen, Hilden, Germany) according to the manufacturer's instructions. The concentration of purified RNA was quantified by BioDrop μLITE (BioDrop, UK).

### Covid-19 Real-Time RT-PCR Assays

Real-time RT-PCR assays for SARS-CoV-2 RNA detection were performed using QuantiNova Probe RT-PCR Kit (Qiagen) in a LightCycler 480 Real-Time PCR System (Roche, Basel, Switzerland) as previously described (Chan, et al., “Improved detection of Zika virus RNA in human and animal specimens by a novel, highly sensitive and specific real-time RT-PCR assay targeting the 5′-untranslated region of Zika virus.” Trop Med Int Health (2017) 22:594-603)). Each 20 μl reaction mixture contained 10 μl of 2× QuantiNova Probe RT-PCR Master Mix, 0.20 μl of QN Probe RT-Mix, 1.6 μl of each 10 μM forward and reverse primer, 0.4 μl of 10 μM probe, 1.2 μl of RNase-free water and 50 of TNA as the template. The thermal cycling condition was 10 min at 45° C. for reverse transcription, 5 min at 95° C. for PCR initial activation, and 45 cycles of 5 s at 95° C. and 30 s at 55° C. The RdRp-P2 assay was performed as previously described (Corman, et al., “Detection of 2019 novel coronavirus (2019-nCoV) by real-time RT-PCR.” Euro Surveill (2020) doi: 10.2807/1560-7917.ES.2020.25.3.2000045)).

### Confirmation of Discrepant Results in Different Covid-19 Real-Time RT-PCR Assays by the LightMix® Modular SARS and Wuhan CoV E-gene Kit with LightCycler Multiplex RNA Virus Master

Discrepant results were confirmed by additional testing with the LightMix® Modular SARS and Wuhan CoV E-gene kit (TIB Molbiol, Berlin, Germany) with LightCycler Multiplex RNA Virus Master (Roche) according to the manufacturer's instructions. Briefly, each 20 μl reaction mixture contained 4 μl of Roche Master, 0.10 of RT Enzyme, 0.5 μl of reagent mix, 10.41 μl of water and 5 μl of TNA as the template. The thermal cycling condition was 5 min at 55° C. for reverse transcription, 5 min at 95° C. for denaturation, and 45 cycles of 5 s at 95° C., 15 s at 60° C. and 15 s at 72° C.

### Statistical Analysis

The Fisher's exact test was used to compare the performance of the assays. P<0.05 was considered statistically significant. Computation was performed using Predictive Analytics Software (v18.0).

### RESULTS

### Design of Covid-19 Real-Time RT-PCR Assays Targeting Different Gene Regions of the SARS-CoV-2 Genome

Three real-time Covid-19 RT-PCR assays targeting the RdRp/Hel, S, and N genes of SARS-CoV-2 were developed (Table 1).

To avoid cross-reactivity with human SARS-CoV, the probes of in these assays were designed to contain 7 to 9 nucleotide differences with those of human SARS-CoV (strains HKU-39849 and GZ50) (FIGS. 1A-1D). In comparison, the probe of the RdRp-P2 assay contained only 3 nucleotide differences with those of human SARS-CoV (strains Frankfurt-1, HKU-39849, and GZ50) (Corman, et al., “Detection of 2019 novel coronavirus (2019-nCoV) by real-time RT-PCR.” Euro Surveill (2020) doi: 10.2807/1560-7917.ES.2020.25.3.2000045) (FIGS. 1A-1D).

### Analytical Sensitivity of the Covid-19 Real-Time RT-PCR Assays

To determine the analytical sensitivity of the Covid-19 assays, their limits of detection were evaluated using viral genomic RNA extracted from culture lysate and clinical specimen. Serial 10-fold dilutions of SARS-CoV-2 RNA extracted from culture lysate were prepared and tested in triplicate with each corresponding assay in two independent runs. The limit of detection of Covid-19-RdRp/Hel, Covid-19-S, and Covid-19-N was 1.8×10° TCID₅₀/ml, while the limit of detection of RdRp-P2 was 1-log higher (1.8×10¹ TCID50/ml) (Table 2). Serial 10-fold dilutions of SARS-CoV-2 RNA extracted from a laboratory-confirmed patient's nasopharyngeal aspirate were also prepared and tested in triplicate with each corresponding assay in two independent runs. The limit of detection of Covid-19-RdRp/Hel and Covid-19-N (10⁻⁵ fold dilution) was 1-log lower than that of Covid-19-S and RdRp-P2 (10⁻⁴ fold dilution) (Table 2).

TABLE 2
Limits of detection of the Covid-19 real-time RT-PCR assays with genomic RNA for SARS-CoV-2 in culture lysate and clinical specimen

| Culture lysate | | | | | | | Clinical specimen | | | | | | |
|---|---|---|---|---|---|---|---|---|---|---|---|---|---|
| | Cp | | | Cp | | | | Cp | | | Cp | | |
| Virus quantity | (Intra-run) | | | (Inter-run) | | | RNA extract | (Intra-run) | | | (Inter-run) | | |
| (TCID₅₀/ml) | Test 1 | Test 2 | Test 3 | Test 1 | Test 2 | Test 3 | (fold dilution) | Test 1 | Test 2 | Test 3 | Test 1 | Test 2 | Test 3 |

Covid-19-RdRp/Hel

| | | | | | | | | | | | | | |
|---|---|---|---|---|---|---|---|---|---|---|---|---|---|
| 1.8 × 10¹ | 34.03 | 33.64 | 33.63 | 33.89 | 33.67 | 33.80 | 10⁻⁴ | 34.86 | 34.97 | 34.79 | 35.34 | 35.20 | 34.89 |
| 1.8 × 10⁰ | 36.90 | 36.43 | 36.41 | 36.94 | 36.61 | 37.25 | 10⁻⁵ | 37.74 | 38.05 | 39.45 | 37.95 | 37.96 | 37.83 |
| 1.8 × 10⁻¹ | 40.00 | 40.00 | 40.00 | 38.52 | 40.00 | − | 10⁻⁶ | − | 40.00 | − | 40.00 | 38.55 | − |
| 1.8 × 10⁻² | − | − | − | − | − | − | 10⁻⁷ | − | − | − | − | − | − |

Covid-19-S

| | | | | | | | | | | | | | |
|---|---|---|---|---|---|---|---|---|---|---|---|---|---|
| 1.8 × 10¹ | 34.88 | 34.96 | 35.08 | 36.32 | 35.94 | 35.64 | 10⁻⁴ | 37.15 | 37.46 | 36.86 | 37.38 | 37.59 | 37.32 |
| 1.8 × 10⁰ | 36.79 | 36.99 | 37.60 | 38.33 | 39.25 | 38.71 | 10⁻⁵ | − | 40.00 | − | − | 40.00 | 40.00 |
| 1.8 × 10⁻¹ | 40.00 | 40.00 | 40.00 | 40.00 | − | − | 10⁻⁶ | − | 40.00 | − | − | − | − |
| 1.8 × 10⁻² | − | − | − | − | − | − | 10⁻⁷ | − | − | − | − | − | − |

Covid-19-N

| | | | | | | | | | | | | | |
|---|---|---|---|---|---|---|---|---|---|---|---|---|---|
| 1.8 × 10¹ | 31.88 | 31.73 | 31.67 | 32.72 | 32.61 | 32.85 | 10⁻⁴ | 35.64 | 35.01 | 35.10 | 35.52 | 35.38 | 35.62 |
| 1.8 × 10⁰ | 34.14 | 34.26 | 34.57 | 35.69 | 35.86 | 35.86 | 10⁻⁵ | 39.16 | 40.00 | 39.09 | 40.00 | 38.12 | 37.12 |
| 1.8 × 10⁻¹ | 38.32 | 37.29 | 36.9 | 40.00 | 38.42 | − | 10⁻⁶ | − | − | 40.00 | − | − | − |
| 1.8 × 10⁻² | − | − | − | − | − | − | 10⁻⁷ | − | − | − | − | − | − |

RdRp-P2

| | | | | | | | | | | | | | |
|---|---|---|---|---|---|---|---|---|---|---|---|---|---|
| 1.8 × 10¹ | 33.46 | 33.74 | 33.49 | 33.53 | 33.45 | 33.46 | 10⁻⁴ | 33.63 | 33.31 | 33.65 | 33.68 | 33.34 | 33.62 |
| 1.8 × 10⁰ | 34.05 | 34.64 | 34.12 | 33.78 | 33.83 | − | 10⁻⁵ | 34.15 | 34.00 | 33.95 | − | − | 34.01 |
| 1.8 × 10⁻¹ | − | − | − | − | − | − | 10⁻⁶ | − | − | − | − | − | − |
| 1.8 × 10⁻² | − | − | − | − | − | − | 10⁻⁷ | − | − | − | − | − | − |

Abbreviations:
+, positive;
−, negative;
Cp, cycle number at detection threshold

Based on these results, the Covid-19-RdRp/Hel and Covid-19-N assays were selected for further evaluation and determined their limits of detection using in vitro viral RNA transcripts.

TABLE 3
Limits of detection of Covid-19 real-time RT-PCR
assays with in vitro RNA transcripts for SARS-CoV-2

| Predicted no. of | No. of positive tests/no. of replicates (%) | | | |
|---|---|---|---|---|
| RNA copies/reaction | Covid-19-RdRp/Hel | | Covid-19-N | |
| 40 | 8/8 | (100.0) | 8/8 | (100.0) |
| 20 | 8/8 | (100.0) | 7/8 | (87.5) |
| 10 | 8/8 | (100) | 7/8 | (87.5) |
| 5 | 3/8 | (37.5) | 5/8 | (62.5) |
| 2.5 | 2/8 | (25.0) | 2/8 | (25.0) |
| 0 | 0/8 | (0.0) | 0/8 | (0.0) |

The limits of detection of the Covid-19-RdRp/Hel and Covid-19-N assays using serial dilutions of in vitro viral RNA transcripts as calculated with probit analysis were 11.2 RNA copies/reaction (95% confidence interval=7.2-52.6 RNA copies/reaction) and 21.3 RNA copies/reaction (95% confidence interval=11.6-177.0 copies/reaction), respectively.

### Cross-Reactivity of the Covid-19-RdRp/Hel and Covid-19-N Assays with Other Human-Pathogenic Coronaviruses and Respiratory Viruses

The SARS-CoV-2 genome is highly similar to that of human SARS-CoV, with an overall ˜82% nucleotide identity (Chan, et al., “Genomic characterization of the 2019 novel human-pathogenic coronavirus isolated from a patient with atypical pneumonia after visiting Wuhan.” Emerg Microbes Infect (2020) 9:221-236)). RT-PCR assays that target gene fragments that are homologous in both viruses may therefore be non-specific. To investigate whether the new Covid-19-RdRp/Hel and Covid-19-N assays cross-react with SARS-CoV, other human-pathogenic coronaviruses, and respiratory viruses, the assays were used to test 17 culture isolates of coronaviruses (SARS-CoV, MERS-CoV, HCoV-OC43, HCoV-229E, and HCoV-NL63), adenovirus, human metapneumovirus, influenza A (H1N1 and H3N2) viruses, influenza B virus, influenza C virus, parainfluenza viruses types 1 to 4, rhinovirus, and respiratory syncytial virus.

TABLE 4
Cross-reactivity between the Covid-19 real-time RT-PCR
assays and other respiratory viruses in cell culture

| Virus | Covid-19-RdRp/Hel | Covid-19-N | RdRp-P2 |
|---|---|---|---|
| SARS-CoV | − | − | + |
| MERS-CoV | − | − | − |
| HCoV-OC43 | − | − | − |
| HCoV-229E | − | − | − |
| HCoV-NL63 | − | − | − |
| Adenovirus | − | − | − |
| hMPV | − | − | − |
| IAV (H1N1) | − | − | − |
| IAV (H3N2) | − | − | − |
| IBV | − | − | − |
| ICV | − | − | − |
| PIV1 | − | − | − |
| PIV2 | − | − | − |
| PIV3 | − | − | − |
| PIV4 | − | − | − |
| Rhinovirus | − | − | − |
| RSV | − | − | − |

Abbreviations: +, positive; −, negative; HCoV, human coronavirus; hMPV, human metapneumovirus; IAV, influenza A virus; IBV, influenza B virus; ICV, influenza C virus; MERS-CoV, Middle East respiratory syndrome coronavirus; PIV, parainfluenza virus; RSV, respiratory syncytial virus; SARS-CoV, severe acute respiratory syndrome coronavirus.

As shown in Table 4, no cross-reactivity with these viruses was found in either assay. Unlike what was previously reported, it was found that the RdRp-P2 assay cross-reacted with SARS-CoV culture lysate (Corman, et al., “Detection of 2019 novel coronavirus (2019-nCoV) by real-time RT-PCR.” Euro Surveill (2020) doi: 10.2807/1560-7917.ES.2020.25.3.2000045)). This cross-reactivity was consistently observed in two independent runs conducted on different days with each run having three technical replicates of each biological replicate (two biological replicates: SARS-CoV strains HKU-39849 and GZ50) and stringent compliance with the published protocol.

### Comparative Performance of the Covid-19-RdRp/Hel and RdRp-P2 for the Detection of SARS-CoV-2 RNA in Different Types of Clinical Specimens

Based on the lower limit of detection of the Covid-19-RdRp/Hel assay than the Covid-19-N assay, the performance of Covid-19-RdRp/Hel assay was then evaluated in the detection of SARS-CoV-2 RNA in clinical specimens and compared it with that of the RdRp-P2 assay. A total of 120 respiratory tract (nasopharyngeal aspirates/swabs, throat swabs, saliva, and sputum) and 153 non-respiratory tract (plasma, urine, and feces/rectal swabs) specimens were collected from 15 patients with laboratory-confirmed Covid-19 (positive nasopharyngeal aspirate/swab, throat swab, or sputum by the RdRp-P2 assay). There were a total of 8 males and 7 females. Their median age was 63 years (range: 37 to 75 years). All of them had clinical features compatible with acute community-acquired atypical pneumonia and radiological evidence of ground-glass lung opacities. At the time of writing, 11 patients were in stable condition, 3 were in critical condition, and 1 patient had succumbed.

Among the 273 specimens collected from these 15 patients, 77 (28.2%) were positive by the RdRp-P2 assay (Table 5).

TABLE 5
Comparison between the Covid-19-RdRp/Hel and RdRp-P2
real-time RT-PCR assays for the detection of SARS-
CoV-2 RNA in different types of clinical specimens
from 15 patients with laboratory-confirmed Covid-19

| | | | | Mean (range) |
|---|---|---|---|---|
| | | | | viral load in |
| | | | | RdRp-P2-negative |
| | | | | but Covid-19-RdRp/ |
| | Covid- | | | Hel-positive |
| | 19-RdRp/ | | P | specimens, RNA |
| Specimen type | Hel | RdRp-P2 | value | copies/ml |
| Respiratory | 102/120 | 73/120 | <0.001 | 4.33 × 10⁴ |
| tract: | (85.0%) | (60.8%) | | (2.85 × 10³ |
| | | | | to 4.71 × 10⁵) |
| NPA/NPS/TS | 30/34 | 22/34 | 0.043 | 1.74 × 10⁴ |
| | (88.2%) | (64.7%) | | (2.85 × 10³ |
| | | | | to 8.40 × 10⁴) |
| Saliva | 59/72 | 38/72 | <0.001 | 5.32 × 10⁴ |
| | (81.9%) | (52.8%) | | (1.74 × 10³ |
| | | | | to 4.71 × 10⁵) |
| Sputum | 13/14 | 13/14 | NS | NA |
| | (92.9%) | (92.9%) | | |
| Non- | 17/153 | 4/153 | 0.005 | 7.06 × 10³ |
| respiratory | (11.1%) | (2.6%) | | (2.21 × 10² |
| tract: | | | | to 1.67 × 10⁴) |
| Plasma | 10/87 | 0/87 | 0.001 | 7.86 × 10³ |
| | (11.5%) | (0.0%) | | (2.21 × 10² |
| | | | | to 1.67 × 10⁴) |
| Urine | 0/33 | 0/33 | NS | NA |
| | (0.0%) | (0.0%) | | |
| Feces/rectal | 7/33 | 4/33 | NS | 4.38 × 10³ |
| swabs | (21.2%) | (12.1%) | | (1.54 × 10³ |
| | | | | to 6.69 × 10³) |
| Total | 119/273 | 77/273 | <0.001 | 3.21 × 10⁴ |
| | (43.6%) | (28.2%) | | (2.21 × 10² |
| | | | | to 4.71 × 10⁵) |

Abbreviations:
NA, not applicable;
NPA, nasopharyngeal aspirate;
NPS, nasopharyngeal swab;
NS, not significant;
TS, throat swab

The new Covid-2019-RdRp/Hel assay was positive for all of these 77 specimens. Additionally, the Covid-2019-RdRp/Hel assay was positive for another 42 [total positive specimens=119/273 (43.6%) by Covid-2019-RdRp/Hel vs 77/273 (28.2%) by RdRp-P2, P<0.001] specimens, including 29/120 (24.2%) respiratory tract specimens and 13/153 (8.5%) non-respiratory tract specimens that were negative by the RdRp-P2 assay. All of these 42/273 (15.4%) additional positive specimens were confirmed to be positive by the LightMix® Modular SARS and Wuhan CoV E-gene kit with the LightCycler Multiplex RNA Virus Master. The mean viral load of these specimens was 3.21×10⁴ RNA copies/ml (range, 2.21×10² to 4.71×10⁵ RNA copies/ml) and was about 6 folds higher in the respiratory tract specimens (4.33×10⁴ RNA copies/ml) than the non-respiratory tract specimens (7.06×10³ RNA copies/ml).

### Cross-Reactivity of the Covid-19-RdRp/Hel Assay with Other Human Pathogenic Coronaviruses and Respiratory Viruses in Nasopharyngeal Aspirates

To investigate whether the Covid-19-RdRp/Hel assay was specific for SARS-CoV-2 in clinical specimens, the assay was used to test 61 archived nasopharyngeal aspirates/swabs and throat swabs that were previously tested by FilmArray RP2 from patients with upper and/or lower respiratory tract symptoms. Among these 61 clinical specimens, 22 were positive and 39 were negative for other human-pathogenic coronaviruses or common respiratory pathogens by FilmArray RP2.

As shown in Table 6, none of these specimens was positive by the Covid-19-RdRp/Hel assay, indicating that the assay was specific for the detection of SARS-CoV-2 RNA in nasopharyngeal aspirates/swabs and throat swabs containing DNA/RNA of other human-pathogenic coronaviruses and respiratory pathogens.

TABLE 6
Lack of cross-reactivity between the Covid-19-RdRp/Hel assay
and other respiratory pathogens in clinical specimens^{a}

| | No. Covid-19-RdRp/Hel-positive |
|---|---|
| FilmArray RP2 result | specimens/No. of total specimens |
| HCoV-OC43 | 0/2 |
| HCoV-HKU1 | 0/1 |
| HCoV-229E | 0/1 |
| Adenovirus | 0/3 |
| IAV | 0/7 |
| PIV | 0/3 |
| Rhinovirus/EV | 0/4 |
| Mycoplasma pneumoniae | 0/1 |
| No pathogen detected | 0/39 |
| Total | 0/61 |

^{a}These included nasopharyngeal aspirates, nasopharyngeal swabs, and throat swabs tested by FilmArray RP2.
Abbreviations: EV, enterovirus; HCoV, human coronavirus; IAV, influenza A virus; PIV, parainfluenza virus.

### DISCUSSION

The positive-sense, single-stranded RNA genome of SARS-CoV-2 is ˜30 kilobases in size and encodes ˜9860 amino acids (Chan, et al., “A familial cluster of pneumonia associated with the 2019 novel coronavirus indicating person-to-person transmission: a study of a family cluster.” Lancet (2020) doi: 10.1016/S0140-6736(20)30154-9. [Epub ahead of print], Chan, et al., “Genomic characterization of the 2019 novel human-pathogenic coronavirus isolated from a patient with atypical pneumonia after visiting Wuhan.” Emerg Microbes Infect (2020) 9:221-236, Lu, et al., “Genomic characterisation and epidemiology of 2019 novel coronavirus: implications for virus origins and receptor binding.” Lancet (2020) doi: 10.1016/S0140-6736(20)30251-8. [Epub ahead of print], Chen, et al., “RNA based mNGS approach identifies a novel human coronavirus from two individual pneumonia cases in 2019 Wuhan outbreak.” Emerg Microbes Infect (2020) 9:313-319)). Like other betacoronaviruses, the SARS-CoV-2 genome is arranged in the order of 5′-replicase (ORF1a/b)-S-E-Membrane-N-poly(A)-3′ (Chan, et al., “Genomic characterization of the 2019 novel human-pathogenic coronavirus isolated from a patient with atypical pneumonia after visiting Wuhan.” Emerg Microbes Infect (2020) 9:221-23)). Traditionally, the preferred targets of coronavirus RT-PCR assays included the conserved and/or abundantly expressed genes such as the structural S and N genes, and the non-structural RdRp and replicase open reading frame (ORF) 1a/b genes (Chan, et al., “Middle East respiratory syndrome coronavirus: another zoonotic betacoronavirus causing SARS-like disease.” Clin Microbiol Rev (2015) 28:465-522, Mackay & Arden., “MERS coronavirus: diagnostics, epidemiology and transmission.” Virol J. (2015) 12:222)). For Covid-19, the protocols of a number of RT-PCR assays used by different institutes have recently been made available online https://www.who.int/emergencies/diseases/novel-coronavirus_2019/technical-guidance/laboratory-guidance). These assays target the ORF1a/b, ORF1b-nsp14, RdRp, S, E, or N genes of SARS-CoV-2 and some are non-specific assays that would detect SARS-CoV-2 and other related betacoronaviruses such as SARS-CoV (Corman, et al., “Detection of 2019 novel coronavirus (2019-nCoV) by real-time RT-PCR.” Euro Surveill (2020) doi:

10.2807/1560-7917.ES.2020.25.3.2000045, Chu, et al., “Molecular Diagnosis of a Novel Coronavirus (2019-nCoV) Causing an Outbreak of Pneumonia.” Clin Chem (2020) doi: 10.1093/clinchem/hvaa029 [Epub ahead of print]).

Importantly, the in-use evaluation data of these assays using a large number of clinical specimens from patients with confirmed Covid-19 are lacking. Three real-time RT-PCR assays that target different gene regions of the SARS-CoV-2 genome were developed and evaluated. The Covid-19-RdRp/Hel assay was highly sensitive and specific for the detection of SARS-CoV-2 RNA in vitro and in Covid-19 patient specimens.

Among the three assays developed in this study, the Covid-19-RdRp/Hel assay has the highest analytical sensitivity (11.2 RNA copies/reaction, 95% confidence interval=7.2-52.6 RNA copies/reaction). The limit of detection with genomic RNA was also very low (1.80 TCID₅₀/ml). Importantly, the Covid-19-RdRp/Hel assay was significantly more sensitive (P≤0.001) than the established RdRp-P2 assay for the detection of SARS-CoV-2 RNA in both respiratory tract and non-respiratory tract clinical specimens. The Covid-19-RdRp/Hel assay detected SARS-CoV-2 RNA in 42/273 (15.4%) additional specimens that were tested negative by the RdRp-P2 assay. These findings are clinically and epidemiologically relevant because asymptomatic and mildly symptomatic cases of Covid-19 have been increasingly recognized and these patients with cryptic pneumonia may serve as a potential source for propagating the epidemic (Chan, et al., “A familial cluster of pneumonia associated with the 2019 novel coronavirus indicating person-to-person transmission: a study of a family cluster.” Lancet (2020) doi: 10.1016/S0140-6736(20)30154-9. [Epub ahead of print], Wei, et al., “Novel Coronavirus Infection in Hospitalized Infants Under 1 Year of Age in China.” JAMA (2020) doi: 10.1001/jama.2020.2131. [Epub ahead of print]). Given the large number of patients (>60,000 cases in China at the time of writing) involved in this expanding epidemic, the additional positivity specimens detected by the Covid-19-RdRp/Hel assay might translate into thousands of specimens that would otherwise be considered as SARS-CoV-2-negative by the less sensitive RdRp-P2 assay.

Regarding the different types of clinical specimens, the Covid-19-RdRp/Hel assay was significantly more sensitive than the RdRp-P2 assay for the detection of SARS-CoV-2 RNA in nasopharyngeal aspirate/swab or throat swab (P=0.043), saliva (P<0.001), and plasma specimens (P=0.001). False-negative results might arise from testing nasopharyngeal aspirate/swabs or throat swabs with low viral loads in Covid-19, SARS, and MERS patients (Chan, et al., “A familial cluster of pneumonia associated with the 2019 novel coronavirus indicating person-to-person transmission: a study of a family cluster.” Lancet (2020) doi: 10.1016/S0140-6736(20)30154-9. [Epub ahead of print], Peiris, et al., “Clinical progression and viral load in a community outbreak of coronavirus-associated SARS pneumonia: a prospective study.” Lancet (2003) 361:1767-1772, Tsang, et al., “Coronavirus-positive nasopharyngeal aspirate as predictor for severe acute respiratory syndrome mortality.” Emerg Infect Dis (2003) 9:1381-1387, Corman, et al, “Viral Shedding and Antibody Response in 37 Patients With Middle East Respiratory Syndrome Coronavirus Infection.” Clin Infect Dis (2016) 62:477-483, Memish, et al., “Respiratory tract samples, viral load, and genome fraction yield in patients with Middle East respiratory syndrome.” J Infect Dis (2014) 210:1590-4)). RT-PCR assays with higher sensitivity, such as the Covid-19-RdRp/Hel assay, might help to reduce the false-negative rate among these specimens which are frequently the only specimens available for establishing the diagnosis of Covid-19. Studies show that saliva has a high concordance rate with nasopharyngeal aspirates for the detection of influenza viral RNA and might also be a suitable specimen for diagnosing Covid-19 (To, et al., “Consistent detection of 2019 novel coronavirus in saliva.” Clin Infect Dis (2020) doi: 10.1093/cid/ciaa149. [Epub ahead of print], To, et al., “Saliva as a diagnostic specimen for testing respiratory virus by a point-of-care molecular assay: a diagnostic validity study.” Clin Microbiol Infect (2019) 25:372-8)). The use of the highly sensitive Covid-19-RdRp/Hel assay to test saliva from suspected cases of Covid-19 might be a simple and rapid way to avoid the need of aerosol-generating procedures during collection of nasopharyngeal aspirates and suction of sputum, especially in regions most heavily affected by the ongoing Covid-19 outbreak where full personal protective equipment are insufficient (Khan, et al., “Novel coronavirus: how the things are in Wuhan.” Clin Microbiol Infect (2020) doi: 10.1016/j.cmi.2020.02.005)). SARS-CoV-2 RNAemia has been reported in a small proportion of Covid-19 patients (Chan, et al., “A familial cluster of pneumonia associated with the 2019 novel coronavirus indicating person-to-person transmission: a study of a family cluster.” Lancet (2020) doi: 10.1016/S0140-6736(20)30154-9. [Epub ahead of print], Huang, et al., “Clinical features of patients infected with 2019 novel coronavirus in Wuhan, China.” Lancet (2020) doi: 10.1016/S0140-6736(20)30183-5. [Epub ahead of print]). However, as shown in the clinical evaluation in which the RdRp-P2 assay was negative for all the 10 plasma specimens that were tested positive by the Covid-19-RdRp/Hel assay, the genuine incidence of SARS-CoV-2 RNAemia might be underestimated by less sensitive RT-PCR assays. Studies show that high serum viral loads in SARS patients were associated with more severe disease as evidenced by higher incidence of oxygen desaturation, need for mechanical ventilation, hepatic dysfunction, and death (Hung et al., “Viral loads in clinical specimens and SARS manifestations.” Emerg Infect Dis (2004) 10:1550-1557)). Thus, serial monitoring of the plasma viral load in Covid-19 patients with the highly sensitive Covid-19-RdRp/Hel assay should be considered to provide prognostic insights and facilitate treatment decisions.

The Covid-19-RdRp/Hel was highly specific and exhibited no cross-reactivity with other common respiratory pathogens in vitro and in nasopharyngeal aspirates. Interestingly, the evaluation showed that the RdRp-P2 assay cross-reacted with SARS-CoV in vitro, which is different from what was previously reported (Corman, et al., “Detection of 2019 novel coronavirus (2019-nCoV) by real-time RT-PCR.” Euro Surveill (2020) doi: 10.2807/1560-7917.ES.2020.25.3.2000045)). This might be due to the small number (n=3) of nucleotide differences between the probe used in the RdRp-P2 assay with at least 3 strains of SARS-CoV (Corman, et al., “Detection of 2019 novel coronavirus (2019-nCoV) by real-time RT-PCR.” Euro Surveill (2020) doi: 10.2807/1560-7917.ES.2020.25.3.2000045)). This cross-reactivity would be especially important for laboratories in areas where SARS-CoV might re-emerge and co-circulate with SARS-CoV-2, as the clinical progressions of SARS and Covid-19 remain incompletely understood at this stage.

In summary, the newly established Covid-19-RdRp/Hel assay is highly sensitive and specific for the detection of SARS-CoV-2 RNA in vitro and in respiratory and non-respiratory tract clinical specimens. The use of Covid-19-RdRp/Hel assay might be especially useful for detecting Covid-19 cases with low viral loads and when testing upper respiratory tract, saliva, and plasma specimens of patients. Development of Covid-19-RdRp/Hel into a multiplex assay which can simultaneously detect other human-pathogenic coronaviruses and respiratory pathogens may further increase its clinical utility in the future.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described.

Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different moieties does not indicate that the listed moieties are obvious one to the other, nor is it an admission of equivalence or obviousness.

1. A nucleic acid probe or primer for the detection of SARS-CoV-2 RdRp/Helicase comprising or consisting of a nucleic acid sequence that hybridizes with
(SEQ ID NO: 1)
CGCATACAGTCTTRCAGGCT,
(SEQ ID NO: 2)
GTGTGATGTTGAWATGACATGGTC,
(SEQ ID NO: 3)
TTAAGATGTGGTGCTTGCATACGTAGAC,
(SEQ ID NO: 4)
GACCATGTCATWTCAACATCACAC,
the reverse complement of any of SEQ ID NOS:1-4;
a nucleic acid sequence having at least 70%, 75%, 80%, 85%, 90%, 95%, 96%, 97%, 98%, or 99% sequence identity to any of the foregoing; or
a nucleic acid sequence having 1, 2, 3, 4, 5, 6, 7, 8, 9, or 10 nucleic acid substitution(s), addition(s), deletion(s), or a combination thereof relative thereto.

2. (canceled)

3. (canceled)

4. The nucleic acid probe of claim 1 comprising a primer pair comprising
a forward primer comprising or consisting of the nucleic acid sequence CGCATACAGTCTTRCAGGCT (SEQ ID NO:1) or a nucleic acid sequence having at least 70%, 75%, 80%, 85%, 90%, 95%, 96%, 97%, 98%, or 99% sequence identity, or a nucleic acid sequence having 1, 2, 3, 4, 5, 6, 7, 8, 9, or 10 nucleic acid substitution(s), addition(s), deletion(s), or a combination thereof relative thereto, and
a reverse primer comprising or consisting of the nucleic acid sequence GTGTGATGTTGAWATGACATGGTC (SEQ ID NO:2) or a nucleic acid sequence having at least 70%, 75%, 80%, 85%, 90%, 95%, 96%, 97%, 98%, or 99% sequence identity; or a nucleic acid sequence having 1, 2, 3, 4, 5, 6, 7, 8, 9, or 10 nucleic acid substitution(s), addition(s), deletion(s), or a combination thereof relative thereto.

5. (canceled)

6. (canceled)

7. (canceled)

8. The nucleic acid probe of claim 1 further comprising one or more fluorescent reporters, one or more quenchers, or a combination thereof, optionally, wherein the one or more fluorescent reporters is a 5′ fluorescent reporter and the one or more quenchers is a 3′ quencher.

9. (canceled)

10. (canceled)

11. A nucleic acid probe or primer for the detection of SARS-CoV-2 Spike (S) comprising or consisting of a nucleic acid sequence that hybridizes with
(SEQ ID NO: 5)
CCTACTAAATTAAATGATCTCTGCTTTACT,
(SEQ ID NO: 6)
CAAGCTATAACGCAGCCTGTA,
(SEQ ID NO: 7)
CGCTCCAGGGCAAACTGGAAAG,
(SEQ ID NO: 8)
TACAGGCTGCGTTATAGCTTG,
the reverse complement of any of SEQ ID NOS:5-8;
a nucleic acid sequence having at least 70%, 75%, 80%, 85%, 90%, 95%, 96%, 97%, 98%, or 99% sequence identity to any of the foregoing or a nucleic acid sequence having 1, 2, 3, 4, 5, 6, 7, 8, 9, or 10 nucleic acid substitution(s), addition(s), deletion(s), or a combination thereof relative thereto.

12. (canceled)

13. (canceled)

14. The nucleic acid probe of claim 11 comprising s primer pair comprising
a forward primer comprising or consisting of the nucleic acid sequence CCTACTAAATTAAATGATCTCTGCTTTACT (SEQ ID NO:5) or a nucleic acid sequence having at least 70%, 75%, 80%, 85%, 90%, 95%, 96%, 97%, 98%, or 99% sequence identity, or a nucleic acid sequence having 1, 2, 3, 4, 5, 6, 7, 8, 9, or 10 nucleic acid substitution(s), addition(s), deletion(s), or a combination thereof relative thereto and
a reverse primer comprising or consisting of the nucleic acid sequence CAAGCTATAACGCAGCCTGTA (SEQ ID NO:6) or a nucleic acid sequence having at least 70%, 75%, 80%, 85%, 90%, 95%, 96%, 97%, 98%, or 99% sequence identity or a nucleic acid sequence having 1, 2, 3, 4, 5, 6, 7, 8, 9, or 10 nucleic acid substitution(s), addition(s), deletion(s), or a combination thereof relative thereto.

15. (canceled)

16. (canceled)

17. (canceled)

18. The nucleic acid probe of claim 11 further comprising one or more fluorescent reporters, one or more quenchers, or a combination thereof; optionally, wherein the one or more fluorescent reporters is a 5′ fluorescent reporter and the one or more quenchers is a 3′ quencher.

19. (canceled)

20. (canceled)

21. A nucleic acid probe or primer for the detection of SARS-CoV-2 Nucleocapsid (N) comprising or consisting of a nucleic acid sequence that hybridizes with
(SEQ ID NO: 9)
GCGTTCTTCGGAATGTCG,
(SEQ ID NO: 10)
TTGGATCTTTGTCATCCAATTTG,
(SEQ ID NO: 11)
AACGTGGTTGACCTACACAGST,
(SEQ ID NO: 12)
CAAATTGGATGACAAAGATCCAA
the reverse complement of any of SEQ ID NOS:9-12, or
a nucleic acid sequence having at least 70%, 75%, 80%, 85%, 90%, 95%, 96%, 97%, 98%, or 99% sequence identity to any of the foregoing, or a nucleic acid sequence having 1, 2, 3, 4, 5, 6, 7, 8, 9, or 10 nucleic acid substitution(s), addition(s), deletion(s), or a combination thereof relative thereto.

22. (canceled)

23. (canceled)

24. The composition of claim 21 comprising a primer pair comprising
a forward primer comprising or consisting of the nucleic acid sequence GCGTTCTTCGGAATGTCG (SEQ ID NO:9) or a nucleic acid sequence having at least 70%, 75%, 80%, 85%, 90%, 95%, 96%, 97%, 98%, or 99% sequence identity, or a nucleic acid sequence having 1, 2, 3, 4, 5, 6, 7, 8, 9, or 10 nucleic acid substitution(s), addition(s), deletion(s), or a combination thereof relative thereto, and
a reverse primer comprising or consisting of the nucleic acid sequence TTGGATCTTTGTCATCCAATTTG (SEQ ID NO:10) or a nucleic acid sequence having at least 70%, 75%, 80%, 85%, 90%, 95%, 96%, 97%, 98%, or 99% sequence identity, or a nucleic acid sequence having 1, 2, 3, 4, 5, 6, 7, 8, 9, or 10 nucleic acid substitution(s), addition(s), deletion(s), or a combination thereof relative thereto.

25. (canceled)

26. (canceled)

27. (canceled)

28. The nucleic acid probe of claim 21 further comprising one or more fluorescent reporters, one or more quenchers, or a combination thereof. optionally, wherein the one or more fluorescent reporters is a 5′ fluorescent reporter and the one or more quenchers is a 3′ quencher

29. (canceled)

30. (canceled)

31. (canceled)

32. (canceled)

33. (canceled)

34. (canceled)

35. (canceled)

36. A method of detecting SARS-CoV-2 in a sample comprising contacting the sample with one or more nucleic acid sequences selected from the group consisting of SEQ ID Nos. 1-12,
optionally, wherein the sample is selected from the group consisting mucus, sputum (processed or unprocessed), bronchial alveolar lavage (BAL), bronchial wash (BW), bodily fluids, cerebrospinal fluid (CSF), urine, tissue (e.g., biopsy material), nasopharyngeal aspirate, nasopharyngeal swab, throat swab, feces, plasma, serum, or whole blood, optionally wherein the sample is processed to isolate nucleic acids.

37. The method of claim 36, wherein the SARS-CoV-2 has a genome comprising the sequence according to GenBank accession no. MN975262, or a variant thereof comprising at least 70%, 75%, 80%, 85%, 90%, 95%, 96%, 97%, 98%, or 99% sequence identity thereto.

38. (canceled)

39. The primer or probe of claim 36, wherein the method of detection comprises analysis by microarray, differential display, RNase protection assay, northern blot, RT-PCR, or a combination thereof.

40. The primer or probe of claim 39, wherein the method of detection comprises target sequence-specific quantitative or realtime RT-PCR.

41. (canceled)

42. (canceled)

43. The method of claim 36 comprising using nucleic acids of a sample as a template for RT-PCR utilizing one or more primer pairs as follows:
(a) a forward primer comprising or consisting of the nucleic acid sequence of SEQ ID NO: 1 or a nucleic acid sequence having at least 70%, 75%, 80%, 85%, 90%, 95%, 96%, 97%, 98%, or 99%sequence identity, and a reverse primer comprising or consisting of the nucleic acid sequence GTGTGATGTTGAWATGACATGGTC (SEQ ID NO: 2) or a nucleic acid sequence having at least 70%, 75%, 80%, 85%, 90%, 95%, 96%, 97%, 98%, or 99%sequence identity;
(b) a forward primer comprising or consisting of the nucleic acid sequence of SEQ ID NO: 5 or a nucleic acid sequence having at least 70%, 75%, 80%, 85%, 90%, 95%, 96%, 97%, 98%, or 99%sequence identity, and a reverse primer comprising or consisting of the nucleic acid sequence CAAGCTATAACGCAGCCTGTA (SEQ ID NO: 6) or a nucleic acid sequence having at least 70%, 75%, 80%, 85%, 90%, 95%, 96%, 97%, 98%, or 99%sequence identity;
(c) a forward primer comprising or consisting of the nucleic acid sequence GCGTTCTTCGGAATGTCG (SEQ ID NO: 9) or a nucleic acid sequence having at least 70%, 75%, 80%, 85%, 90%, 95%, 96%, 97%, 98%, or 99%sequence identity, and a reverse primer comprising or consisting of the nucleic acid sequence TTGGATCTTTGTCATCCAATTTG (SEQ ID NO: 10) or a nucleic acid sequence having at least 70%, 75%, 80%, 85%, 90%, 95%, 96%, 97%, 98%, or 99%sequence identity.
optionally in combination one or more probes as follows: the nucleic acid sequence of SEQ ID NO: 3, SEQ ID NO:7 or SEQ ID NO:11; a nucleic acid sequence having at least 70%, 75%, 80%, 85%, 90%, 95%, 96%, 97%, 98%, or 99% sequence identity to SEQ ID NO: 3, SEQ ID NO:7 or SEQ ID NO:11, or a nucleic acid sequence having 1, 2, 3, 4, 5, 6, 7, 8, 9, or 10 nucleic acid substitution (s), addition (s), deletion (s), or a combination thereof relative thereto.

44. (canceled)

45. The method of claim 43, wherein the detection of SARS-CoV-2 comprises detection of one or more amplicons formed by PCR utilizing one or more of the primer pairs or composition.

46. The method of claim 43, wherein the sample is a biological sample.

47. The method of claim 46, wherein the biological sample is selected from mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood.

48. The method of claim 47, wherein the sample is processed to expose or isolate the nucleic acids.

49. The method of claim 43, wherein the sample is isolated from a subject suspected of having SARS-CoV-2.

50. The method of claim 36, wherein the primer, probe, method is more sensitive, selective, or a combination thereof for SARS-CoV-2 relative to one or more other human- and/or non-human pathogenic coronaviruses and/or respiratory pathogens, such as SARS-CoV, MERS-CoV, HCoV-OC43, HCoV-229E, HCoV-NL63, adenovirus, human metapneumovirus, influenza A (H1N1 and H3N2) viruses, influenza B virus, influenza C virus, parainfluenza viruses types 1 to 4, rhinovirus, respiratory syncytial virus, Bat-SL-CoV, and combinations thereof.

51. The method of claim 36, further comprising diagnosing a subject with SARS-CoV 2, wherein detection of SARS-CoV-2 in the sample indicates the subject has SARS-CoV-2.

52. (canceled)
